SEQUENCE LISTING (10 sequences)

SEQ ID NO: 1 - DNA, 43 bp, Artificial Sequence
  Oligonucleotide primer heavy chain, 5′ end
caccatggagactgggctgcgctggcttctcctggtcgctgtg

SEQ ID NO: 2 - DNA, 37 bp, Artificial Sequence
  Oligonucleotide primer heavy chain, 3′ end
ctcccgctctccgggtaaatgagcgctgtgccggcga

SEQ ID NO: 3 - DNA, 36 bp, Artificial Sequence
  Oligonucleotide primer light chain kappa, 5′end
caggcaggacccagcatggacacgagggcccccact

SEQ ID NO: 4 - DNA, 34 bp, Artificial Sequence
  Oligonucleotide primer L kappa, 3′end
tcaataggggtgactgttagagcgagacgcctgc

SEQ ID NO: 5 - DNA, 20 bp, Artificial Sequence
  Oligonucleotide primer Primer 1
aattcacattgattattgag

SEQ ID NO: 6 - DNA, 40 bp, Artificial Sequence
  Oligonucleotide primer Primer 2
cagcgcagcccagtctccatcccgtaagcagtgggttctc

SEQ ID NO: 7 - DNA, 40 bp, Artificial Sequence
  Oligonucleotide primer Primer 3
ccgggtaaatgagcgctgtggtttaaacccgctgatcagc

SEQ ID NO: 8 - DNA, 20 bp, Artificial Sequence
  Oligonucleotide primer Primer 4
aagccatagagccgaccgca

SEQ ID NO: 9 - DNA, 11 bp, Artificial Sequence
  Oligonucleotide primer
cggaacaaggg

SEQ ID NO: 10 - DNA, 11 bp, Artificial Sequence
  Oligonucleotide primer
cggaacaaggg

(19) United States
(12) Patent Application Publication
(10) Pub. No.: US 2004/0067496 A1
(43) Pub. Date: Apr. 8, 2004

(54) System for production and screening of monoclonal antibodies

(76) Inventors: Robert Pytela, San Francisco, CA (US); Dongxiao Zhang, Moraga, CA (US); Weimin Zhu, San Francisco, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/266,387

(22) Filed: Oct. 7, 2002

(51) Int. Cl. C12Q 1/68; G01N 33/53; G01N 33/567; C12P 21/02; C12N 5/06

(52) U.S. Cl. 435/6; 435/7.2; 435/326; 435/69.1; 435/320.1

(57) ABSTRACT

The invention provides methods for producing a monoclonal antibody in a host cell. The methods involve introducing linear expression cassettes containing coding sequences for immunoglobulin heavy and light chains into a host cell and expressing a monoclonal antibody. In most embodiments, the immunoglobulin heavy and light chains are both derived from a single antibody-producing cell. The invention further provides methods for producing a plurality of monoclonal antibodies, and methods of screening a plurality of monoclonal antibodies to identify a monoclonal antibody of interest and its encoding nucleic acid. Also provided by the invention are host cells containing monoclonal antibody-encoding sequences, and libraries of monoclonal antibodies for use in screening methods. The invention further provides kits for carrying out the subject methods. The subjects systems, methods and kits find use in a variety of different industrial, medical and research applications.

### FIELD OF THE INVENTION

The field of this invention is monoclonal antibodies. The invention relates to methods of making monoclonal antibodies, particularly methods that do not involve the production of hybridoma cell lines.

### BACKGROUND OF THE INVENTION

Monoclonal antibodies (Mabs) have been available for over 25 years and have revolutionalized biomedical research, especially in the areas of disease diagnosis and the treatment of infection and diseases.

The conventional method for the production of monoclonal antibodies involves hybridomas (Köhler & Milstein, Nature 256:495-7, 1975). In this method, splenic or lymphocyte cells from a mammal which has been injected with antigen are fused with a tumor cell line, thus producing hybrid cells. These hybrid cells, or “hybridomas”, are both immortal and capable of producing the genetically coded antibody of a B cell. To select a hybridoma producing a single antibody, the hybridomas made by cell fusion are segregated by selection, dilution, and regrowth until a single genetically pure antibody-expressing cell line is selected. Because hybridomas produce homogeneous antibodies against a desired antigen, they are called “monoclonal ” antibodies. Hybridoma technology has primarily been focused on the fusion of murine lines, but also human-human hybridomas, human-murine hybridomas, rabbit-rabbit hybridomas and other xenogenic hybrid combinations have been made.

Monoclonal antibodies produced by hybridomas, while clearly preferable to polyclonal antibodies because of their specificity and affinity, suffer from certain disadvantages (see Winter & Milstein, 1991 Nature 349:293-9, 1991; Babcook et al., Proc Natl Acad Sci 93:7843-8, 1996).

Firstly, hybridoma lines producing monoclonal antibodies tend to be unstable and may alter the structure of the antibody produced or stop producing antibody altogether (Kohler et al Proc. Natl. Acad. Sci 77:2197; Morrison J. Immunol 123: 793, 1980). In many cases, hybridomas require repeated subculturing to stabilize antibody production.

Secondly, the hybridoma approach only allows for the immortalization of those cells that are capable of fusing with myeloma cells, and, as such, a very small fraction of the specific antibody-forming cells are available for hybridoma production. It is poorly understood what determines the ability of a B cell to fuse with a myeloma to form an antibody-secreting hybridoma, but it is likely that this capacity is predominantly found in proliferating B cell progenitor cells present in the germinal centers of spleen and lymph nodes. This cell type represents only a narrow window in B cell development (see e.g. Calame, Nat Immunol. 2: 1103-8, 2001), and, as such, a large proportion of B cells are not even competent for fusion with a myeloma. Since cell fusion is itself a very inefficient process, in many cases only a few hundred hybridomas can be produced from a whole animal spleen.

Thirdly, since making hybridoma cells and screening the cells for antibody production requires extensive tissue culture and handling of cells and reagents, a further limitation of the approach is that it is labor intensive and time-consuming. As such, the production of monoclonal antibodies is usually costly and low-throughput.

Fourthly, since conventional hybridoma technology requires sacrifice of the animal in order to obtain its spleen or a lymph node, the methods are unsuitable for production of human monoclonal antibodies.

As such, there is a great need for improved methods for monoclonal antibody production. The present invention addresses this, and other, needs.

Literature

References of interest include U.S. Pat. Nos. 5,472,868, 4,977,081 and 4,859,595 and publications Knight and Becker, Cell 60: 963-970 (1990), Becker and Knight, Cell 63:987-997 (1990), Babcook et al., Proc Natl Acad Sci 93:7843-8 (1996), Bos et al., Eur J Immunol 24:59-65 (1994), Calame, Nat Immunol 2:1103-8 (2001), de Wildt et al., 207:61-7 (1997), Durocher et al., Nucleic Acids Res 30:E9 (2002), Huse et al Biotechnology 24:517-23 (1992), Marks et al., J Mol Biol 222:581-97 (1991), Ochsenbein et al., Proc Natl Acad Sci 97:13263-8 (2000), Sehgal et al J Immunol 161:5347-56 (1998), Slifka et al Curr Opin Immunol 10:252-8 (1998), Spieker-Polet et al., Proc Natl Acad Sci 92:9348-52 (1995), Takahashi et al., J Biotechnol 49:201-10 (1996); Friedmann et al., J. Immunology 152:632-641, Sastry et at., Proc Natl. Acad. Sci. 86:5728-5732, 1989; and Orlandi et al., Proc. Natl. Acad. Sci. 86:3833-3837, 1989.

### SUMMARY OF THE INVENTION

The invention provides methods for producing a monoclonal antibody in a host cell. The methods involve introducing linear expression cassettes containing coding sequences for immunoglobulin heavy and light chains into a host cell and expressing a monoclonal antibody. In most embodiments, the immunoglobulin heavy and light chains are both derived from a single antibody-producing cell. The invention further provides methods for producing a plurality of monoclonal antibodies, and methods of screening a plurality of monoclonal antibodies to identify a monoclonal antibody of interest and its encoding nucleic acid. Also provided by the invention are host cells containing monoclonal antibody-encoding sequences, and libraries of monoclonal antibodies for use in screening methods. The invention further provides kits for carrying out the subject methods. The subjects systems, methods and kits find use in a variety of different industrial, medical and research applications.

### BRIEF DESCRIPTION OF THE DRAWINGS

FIGS. 1A-1D. Cell-ELISA analysis of anti-beta-6 integrin antibody activity in experimental cultures.

### DEFINITIONS

The terms “antibody” and “immunoglobulin” are used interchangeably herein. These terms are well understood by those in the field, and refer to a protein consisting of one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the terms are Fab′, Fv, F(ab′)₂, and or other antibody fragments that retain specific binding to antigen.

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988); Bird et al., Science, 242, 423-426 (1988); see Hood et al., “Immunology”, Benjamin, N.Y., 2nd ed. (1984), and Hunkapiller and Hood, Nature, 323, 15-16 (1986)).

An immunoglobulin light or heavy chain variable region consists of a “framework” region interrupted by three hypervariable regions, also called “complementarity determining regions” or CDRs. The extent of the framework region and CDRs have been precisely defined (see, “Sequences of Proteins of Immunological Interest,” E. Kabat et al., U.S. Department of Health and Human Services, (1983)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

Chimeric antibodies are antibodies whose light and heavy chain genes have been constructed, typically by genetic engineering, from antibody variable and constant region genes belonging to different species. For example, the variable segments of the genes from a rabbit monoclonal antibody may be joined to human constant segments, such as gamma 1 and gamma 3. An example of a therapeutic chimeric antibody is a hybrid protein composed of the variable or antigen-binding domain from a rabbit antibody and the constant or effector domain from a human antibody (e.g., the anti-Tac chimeric antibody made by the cells of A.T.C.C. deposit Accession No. CRL 9688), although other mammalian species may be used.

As used herein, unless otherwise indicated or clear from the context, antibody domains, regions and fragments are accorded standard definitions as are well known in the art. See, e.g., Abbas, A. K., et al., (1991) Cellular and Molecular Immunology, W. B. Saunders Company, Philadelphia, Pa.

As used herein, the term “humanized antibody” or “humanized immunoglobulin” refers to an antibody comprising one or more CDRs from an animal antibody, the antibody having being modified in such a way so as to be less immunogenic in a human than the parental animal antibody. An animal antibody can be humanized using a number of methodologies, including chimeric antibody production, CDR grafting (also called reshaping), and antibody resurfacing.

As used herein, the term “murinized antibody” or “murinized immunoglobulin” refers to an antibody comprising one or more CDRs from an animal antibody, the antibody having being modified in such a way so as to be less immunogenic in a mouse than the parental animal antibody. An animal antibody can be murinized using a number of methodologies, including chimeric antibody production, CDR grafting (also called reshaping), and antibody resurfacing.

It is understood that the humanized antibodies designed and produced by the present method may have additional conservative amino acid substitutions which have substantially no effect on antigen binding or other antibody functions. By conservative substitutions is intended combinations such as gly, ala; val, ile, leu; asp, glu; asn, gln; ser, thr; lys, arg; and phe, tyr.

As used herein, the terms “determining,” “measuring,” and “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

As used herein the term “isolated,” when used in the context of an isolated antibody, refers to an antibody of interest that is at least 60% free, at least 75% free, at least 90% free, at least 95% free, at least 98% free, and even at least 99% free from other components with which the antibody is associated with prior to purification.

A “coding sequence” or a sequence that “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can-include, but is not limited to, cDNA from viral, procaryotic or eucaryotic mRNA, genomic DNA sequences from viral or procaryotic DNA, and synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences that are immunologically identifiable with a polypeptide encoded by the sequence.

“Operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of a coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells, which can be accomplished by genomic integration of all or a portion of the vector, or transient or inheritable maintenance of the vector as an extrachromosomal element. Thus, the term includes cloning, and expression vehicles, as well as integrating vectors.

An “expression cassette” comprises any nucleic acid construct capable of directing the expression of a gene/coding sequence of interest, which is operably linked to a promoter of the expression cassette. Such cassettes can be constructed into a “vector,” “vector construct,” “expression vector,” or “gene transfer vector,” in order to transfer the expression cassette into target cells. Thus, the term includes cloning and expression vehicles, as well as viral vectors.

Techniques for determining nucleic acid and amino acid “sequence identity” are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10 ; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate +PIR. Details of these programs can be found at the following internet address:

http://www.ncbi.nlm.gov/cgi-bin/BLAST.

Alternatively, homology can be determined by hybridization of polynucleotides under conditions that form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80%-85%, preferably at least about 85%-90%, more preferably at least about 90%-95%, and most preferably at least about 95%-98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., infra; DNA Cloning, supra; Nucleic Acid Hybridization, supra.

Two nucleic acid fragments are considered to “selectively hybridize” as described herein when they detectably pair with each other. The degree of sequence identity between two nucleic acid molecules affects the efficiency and strength of hybridization events between such molecules. A partially identical nucleic acid sequence will at least partially inhibit a completely identical sequence from hybridizing to a target molecule. Inhibition of hybridization of the completely identical sequence can be assessed using hybridization assays that are well known in the art (e.g., Southern blot, Northern blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. A nucleic acid molecule that is capable of hybridizing selectively to a target sequence under “moderately stringent” conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/target hybridization where the probe and target have a specific degree of sequence identity, can be determined as is known in the art (see, for example, Nucleic Acid Hybridization. A Practical Approach, editors B. D. Hames and S. J. Higgins, (1985) Oxford; Washington, D.C.; IRL Press).

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.). An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5× Denhardt's solution, 10% dextran sulfate, and 20 mg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed to identify nucleic acids of this particular embodiment of the invention.

A first polynucleotide is “derived from” a second polynucleotide if it has the same or substantially the same nucleotide sequence as a region of the second polynucleotide, its cDNA, complements thereof, or if it displays sequence identity as described above. A first polynucleotide may be derived from a second polynucleotide if the first polynucleotide is used as a template for, e.g. amplification of the second polynucleotide.

A first polypeptide is “derived from” a second polypeptide if it is (i) encoded by a first polynucleotide derived from a second polynucleotide, or (ii) displays sequence identity to the second polypeptides as described above. The term “unit dosage form” as used herein refers to physically discrete units suitable as unitary dosages for subjects (e.g., animals, usually humans), each unit containing a predetermined quantity of an agent, e.g. an antibody in an amount sufficient to produce the desired effect in association with a pharmaceutically acceptable diluent, carrier or vehicle. The specifications for the novel unit dosage forms of the present invention will depend on a variety of factors including, but not necessarily limited to, the particular agent employed and the effect to be achieved, and the pharmacodynamics associated with each compound in the host.

A polynucleotide is “derived from” a particular cell if the polynucleotide was obtained from the cell. A polynucleotide may also be “derived from” a particular cell if the polynucleotide was obtained from the progeny of the cell, as long as the polynucleotide was present in the original cell. As such, a single cell may be isolated and cultured, e.g. in vitro, to form a cell culture. A nucleotide isolated from the cell culture is “derived from” the single cell, as long as the nucleic acid was present in the isolated single cell.

The terms “treatment” “treating” and the like are used herein to refer to any treatment of any disease or condition in a mammal, e.g. particularly a human or a mouse, and includes: a) preventing a disease, condition, or symptom of a disease or condition from occurring in a subject which may be predisposed to the disease but has not yet been diagnosed as having it; b) inhibiting a disease, condition, or symptom of a disease or condition, e.g., arresting its development and/or delaying its onset or manifestation in the patient; and/or c) relieving a disease, condition, or symptom of a disease or condition, e.g., causing regression of the condition or disease and/or its symptoms.

The terms “subject,” “host,” “patient,” and “individual” are used interchangeably herein to refer to any mammalian subject for whom diagnosis or therapy is desired, particularly humans. Other subjects may include cattle, dogs, cats, guinea pigs, rabbits, rats, mice, horses, and so on.

Before the present subject invention is described further, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an antibody” includes a plurality of such antibodies and reference to “a variable domain” includes reference to one or more variable domains and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF PREFERRED EMBODIMENTS

The invention provides methods for producing a monoclonal antibody in a host cell. The methods involve introducing linear expression cassettes containing coding sequences for immunoglobulin heavy and light chains into a host cell and expressing a monoclonal antibody. In most embodiments, the immunoglobulin heavy and light chains are both derived from a single antibody-producing cell. The invention further provides methods for producing a plurality of monoclonal antibodies, and methods of screening a plurality of monoclonal antibodies to identify a monoclonal antibody of interest and its encoding nucleic acid. Also provided by the invention are host cells containing monoclonal antibody-encoding sequences, and libraries of monoclonal antibodies for use in screening methods. The invention further provides kits for carrying out the subject methods. The subjects systems, methods and kits find use in a variety of different industrial, medical and research applications.

In further describing the subject invention, the methods of the invention will be described first, followed by a review of the methods and representative applications in which the subject systems find use and kits that include the subject systems.

Methods for Producing a Monclonal Antibody

The invention provides methods for producing a monoclonal antibody. In general, the methods involve the following steps: a) operably linking a first nucleic acid encoding an immunoglobulin-heavy chain variable domain isolated from an antibody-producing cell to a second nucleic acid to form a linear expression cassette for an immunoglobulin heavy chain; b) operably linking a third nucleic acid encoding an immunoglobulin light chain variable domain isolated from the same antibody-producing cell to a fourth nucleic acid to form a linear expression cassette for an immunoglobulin light chain; (c) introducing the linear expression cassettes into a host cell; and (d) incubating the host cell under conditions sufficient to provide for production of a monoclonal antibody.

Antibody-Producing Cells

An antibody-producing cell is a cell that produces antibodies. Such cells are typically cells involved in a mammalian immune response, such as a B-lymphocyte or its progeny including the plasma cell, and usually produce immunoglobulin heavy and light chains that have been “naturally paired” by the immune system of the host. These cells may either secrete antibodies (antibody-secreting cells) or maintain antibodies on the surface of the cell without secretion into the cellular environment. Also encompassed by the term antibody producing cell is a hybridoma cell that expresses an antibody.

An antibody-producing cell may be obtained from an animal which has not been immunized with a selected antigen, which has been immunized with a selected antigen, or which has developed an immune response to an antigen as a result of disease or condition. Animals may be immunized with a selected antigen using any of the techniques well known in the art suitable for generating an immune response (see Handbook of Experimental Immunology D. M. Weir (ed.), Vol 4, Blackwell Scientific Publishers, Oxford, England, 1986). Within the context of the present invention, the phrase “selected antigen” includes any substance to which an antibody may be made, including, among others, proteins, carbohydrates, inorganic or organic molecules, transition state analogs that resemble intermediates in an enzymatic process, nucleic acids, cells, including cancer cells, cell extracts, pathogens, including living or attenuated viruses, bacteria and the like. As will be appreciated by one of ordinary skill in the art, antigens which are of low immunogenicity may be accompanied with an adjuvant or hapten in order to increase the immune response (for example, complete or incomplete Freund's adjuvant) or with a carrier such as keyhole limpet hemocyanin (KLH).

Many warm-blooded animals, in particular mammals such as humans, rabbits; mice, rats, sheep, cows or pigs and aves such as chickens and turkeys; may be used in order to obtain antibody-forming cells. However, rabbits and mice are generally preferred because of their ease in handling, well-defined genetic traits, and the fact that they may be readily sacrificed. Procedures for immunizing animals are well known in the art, and are described in Harlow et al,. (Antibodies: A Laboratory Manual, First Edition (1988) Cold Spring Harbor, N.Y.). Antibody-producing cells may also be obtained from a subject which has generated the cells during the course of a selected disease or condition. For instance, antibody-producing cells from a human with a disease of unknown cause, such as rheumatoid arthritis, may be obtained and used in an effort to identify antibodies which have an effect on the disease process or which may lead to identification of an etiological agent or body component that is involved in the cause of the disease. Similarly, antibody-producing cells may be obtained from subjects with disease due to known etiological agents such as malaria or AIDS. These antibody-producing cells may be derived from the blood, lymph nodes or bone marrow, as well as from other diseased or normal tissues. Antibody-producing cells may also be prepared from blood collected with an anticoagulant such as heparin or EDTA. The antibody-producing cells may be further separated from erythrocytes and polymorphs using standard procedures such as centrifugation with Ficoll-Hypaque (Pharmacia, Uppsula, Sweden). Antibody-producing cells may also be prepared from solid tissues such as lymph nodes or tumors by dissociation with enzymes such as collagenase and trypsin in the presence of EDTA.

Antibody-producing cells may also be obtained by culture techniques such as in vitro immunization. Examples of such methods are described Reading in Methods in Enzymology (21:18-33 J. J. Langone, H. H. van Vunakis (eds.), Academic Press Inc., N.Y.; 1986). Briefly, a source of antibody-producing cells, such as a suspension of spleen or lymph node cells, or peripheral blood mononuclear cells are cultured in medium such as RPMI 1640 with 10% fetal bovine serum and a source of the substance against which it is desired to develop antibodies. This medium may be additionally supplemented with amounts of substances known to enhance antibody-forming cell activation and proliferation such as lipopolysaccharide or its derivatives or other bacterial adjuvants or cytokines such as IL-1, IL-2, IL-4, IL-5, IL-6, GM-CSF, and IFN-gamma. To enhance immunogenicity, the selected antigen may be coupled to the surface of cells, for example, spleen cells, by conventional techniques such as the use of biotin/avidin.

Once a suitable animal containing an antibody-producing cell has been identified or produced, spleen, lymph node or bone marrow tissue is typically removed, and a cell suspension of antibody-producing cells is prepared using techniques well known in the art. In most embodiments, this suspension is a single cell suspension, techniques for the preparation, of which are well known in the art, e.g., Harlow et al,. (Antibodies. A Laboratory Manual, First Edition (1988) Cold Spring Harbor, N.Y.).

Antibody-producing cells may be enriched from the single cell suspension by methods based upon the size or density of the antibody-forming cells relative to other cells. An example of the use of Percoll to separate cells according to density is described by van Mourik and W. P. Zeizlmaker in Methods in Enzymology 121:174-182 (J. J. Langone, H. H. van Vunakis (eds.), Academic Press Inc., N.Y.). Gradients of varying density of solutions of bovine serum albumin can also be used to separate cells according to density. (See N. Moav and T. N. Harris, J. Immunol 105:1512, 1970; see also Raid, D. J. in SELECTED METHODS IN CELLULAR IMMUNOLOGY, B. Mishell and S. Shiigi (eds.), W. H. Freeman and Co., San Francisco, 1987). Antibody-producing cells may also be enriched and plated using other methods. Exemplary antibody-producing cell enrichment methods include performing flow cytometry (FACS) of cell populations obtained from rabbit spleen, bone marrow, lymph node or other lymph organs, e.g., through incubating the cells with labeled anti-rabbit IgG and sorting the labeled cells using a FACSVantage SE cell sorter (Becton-Dickinson, San Jose, Calif.). In many embodiments, single or nearly single antibody-producing cells are deposited in microtiter plates. If the FACS system is employed, sorted cells may be deposited after enrichment directly into a microtiter plate. Unenriched cells, or cells enriched by any of the above methods may be deposited into a microtiter plate at a limiting dilution (Harlow et al,. (Antibodies: A Laboratory Manual, First Edition (1988) Cold Spring Harbor, N.Y.) to ensure single cell deposition.

In certain embodiments, the antibody-producing cells are not further selected based on the specificity of the antibodies that are expressed by the antibody-producing cells. As such, typically, antibody producing cells are used directly after enrichment, and are not subject to any further purification or selection based on the reactivity or specificity of the antibodies being expressed by the antibody producing cell. In these embodiments, the antibody-producing cells are of “unknown” specificity because the antigen (i.e. the protein, pathogen, peptide, cell, cell extract, nucleic acid, carbohydrate etc.) to which the antibodies of the antibody producing cells bind is not determined.

Optionally, the antibody-producing cells are then cultured (i.e. grown in media that supports at least one, at least 5 or at least 10 or more cell divisions of the cell) by methods known to one of skill in the art after they have been deposited (see e.g. WO 01/55216). As such, the nucleic acids described below may be obtained from the progeny of a single antibody-producing cell. In certain embodiments, however, the antibody cells are not cultured after they are deposited.

In most embodiments of the invention, a single cell is deposited. However, in certain other embodiments, an average of about 1, more than about 2, more than about 5, more than about 10, more than about 20, more than about 50 or more than about 100 antigen producing cells are usually deposited.

Immunoglobulin Heavy Chain-Encoding Nucleic Acids

In many embodiments of the invention, a nucleic acid encoding an immunoglobulin heavy chain variable domain is isolated from an antibody producing cell. This nucleic acid may encode a heavy chain variable domain alone, or may encode a larger fragment of an immunoglobulin heavy chain, such as a heavy chain variable domain and part of the heavy chain constant region, or an entire immunoglobulin heavy chain, optionally including the N-terminal methionine and secretion signal of the immunoglobulin heavy chain.

The immunoglobulin heavy chain-encoding nucleic acid, once isolated from the cell, is operably linked to an expression polynucleotide that will allow for expression, and optionally secretion of a functional immunoglobulin heavy chain from a host cell. In most embodiments, therefore, the expression polynucleotide may encode an appropriate region of an immunoglobulin heavy chain, such as a constant domain or a secretion signal peptide to allow a functional immunoglobulin heavy chain to be expressed, and optionally secreted. For example, if the nucleic acid isolated from a cell encodes an immunoglobulin heavy chain variable domain without a constant domain, an appropriate constant domain-encoding polynucleotide, which will optionally encode a secretion signal peptide, will be operably linked to the nucleic acid. In some embodiments, a nucleic acid encoding an entire immunoglobulin heavy chain, including the N-terminal methionine, will be isolated from a cell. In these embodiments, the expression polynucleotide will usually not encode any part of an immunoglobulin heavy chain.

In some embodiments, where the operably linked expression polynucleotide encodes an appropriate region of an immunoglobulin heavy chain, the polynucleotide may encode a region from a different species as compared to the species from which the cell is derived. For example, the appropriate region may be a human, mouse, rabbit or an appropriate region from any mammalian species. If a humanized monoclonal antibody is desired, human sequences may be chosen, whereas if a murinized monoclonal antibody is desired, mouse sequences may be chosen.

In most embodiments, the immunoglobulin heavy chain-encoding nucleic acid does not encode any part of a viral-derived polypeptide, and encodes a secretion signal peptide sufficient for secretion of the expressed immunoglobulin heavy chain into culture medium.

Immunoglobulin Light Chain-Encoding Nucleic Acids

In many embodiments of the invention, a nucleic acid encoding an immunoglobulin light chain variable domain is isolated from the same antibody-producing cell as the immunoglobulin heavy chain-encoding nucleic acid. This light chain-encoding nucleic acid may encode a light chain variable domain alone, or may encode a larger fragment of an immunoglobulin light chain, such as a light chain variable domain and part of the light chain constant region, or an entire immunoglobulin light chain, optionally including the N-terminal methionine and secretion signal of the immunoglobulin light chain.

The immunoglobulin light chain-encoding nucleic acid, once isolated from the cell, is operably linked to an expression polynucleotide that will allow for expression, and optionally secretion of a functional immunoglobulin light chain from a host cell. In most embodiments, therefore, the expression polynucleotide may encode an appropriate region of an immunoglobulin light chain, such as a constant domain or a secretion signal peptide to allow a functional immunoglobulin light chain to be expressed, and optionally secreted. For example, if the nucleic acid isolated from the cell encodes an immunoglobulin light chain variable domain without a constant domain, an appropriate constant domain-encoding polynucleotide, which will optionally encode a secretion signal peptide, will be operably linked to the nucleic acid. In some embodiments, a nucleic acid encoding an entire immunoglobulin light chain, including the N-terminal methionine, will be isolated from a cell. In these embodiments, the expression polynucleotide will usually not encode any part of an immunoglobulin light chain.

In some embodiments, where the operably linked expression polynucleotide encodes an appropriate region of an immunoglobulin light chain, the polynucleotide may encode a region from a different species as compared to the species from which the cell is derived. For example, the appropriate region may be a human, mouse, rabbit or an appropriate region from any mammalian species. If a humanized monoclonal antibody is desired, human sequences may be chosen, whereas if a murinized monoclonal antibody is desired, mouse sequences may be chosen.

In most embodiments, the immunoglobulin light chain-encoding nucleic acid does not encode any part of a viral-derived polypeptide, and encodes a secretion signal peptide sufficient for secretion of the expressed immunoglobulin heavy chain into culture medium.

Expression Cassettes

The expression polynucleotide further provides expression cassettes for expression of the immunoglobulin heavy and light chains in a host cell. In most embodiments, each expression cassette is more than about 0.5 kb in length, more than about 1.0 kb in length, more than about 1.5 kb in length, more than about 2 kb in length, more than about 4 kb in length, more than about 5 kb in length, and is usually less than 10 kb in length. The expression cassette may be linear, or encompassed in a circular vector.

Each of the heavy and light chain expression polynucleotides described above will typically further include expression control DNA sequences operably linked to the immunoglobulin coding sequences to form heavy and light chain expression cassettes. In some embodiments, the expression control sequences will be eukaryotic promoter capable of directing expression of the immunoglobulin heavy or light chain polypeptide in eukaryotic host cells. In certain embodiments, a human cytomegalovirus (HCMV) promoter and/or enhancer and/or terminator is used to direct expression of the polypeptides in mammalian cells. Suitable promoters, terminators, and translational enhancers suitable for expression of immunoglobulin heavy and light chains are known in the art, and many are discussed in Ausubel, et al, (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.). Suitable promoters include SV40 elements, as described in Dijkema et al., EMBO J. (1985) 4:761; transcription regulatory elements derived from the LTR of the Rous sarcoma virus, as described in Gorman et al., Proc. Nat'l Acad. Sci USA (1982) 79:6777; transcription regulatory elements derived from the LTR of human cytomegalovirus (CMV), as described in Boshart et al., Cell (1985) 41:521; hsp70 promoters, (Levy-Holtzman, R. and I. Schechter (Biochim. Biophys. Acta (1995) 1263: 96-98) Presnail, J. K. and M. A. Hoy, (Exp. Appl. Acarol. (1994) 18: 301-308)) and the like.

In many embodiments of the invention, the heavy and light chain expression cassettes are linear expression cassettes, or are present on a circular nucleic acid (e.g. a circular vector, for example a plasmid). Linear expression cassettes are typically not inserted into a circular vector and are not otherwise associated with vector sequences such as an origin of replication, or vector backbone. In certain embodiments, however, the linear expression cassette may also provide for expression of a selectable marker. Suitable vectors and selectable markers are well known in the art and discussed in Ausubel, et al, (Short Protocols in Molecular Biology, 3rd ed, Wiley & Sons, 1995) and Sambrook, et al, (Molecular Cloning: A Laboratory Manual, Third Edition, (2001) Cold Spring Harbor, N.Y.). A variety of different genes have been employed as selectable markers, and the particular gene employed in the subject vectors as a selectable marker is chosen primarily as a matter of convenience. Known selectable marker genes include: the thimydine kinase gene, the dihydrofolate reductase gene, the xanthine-guanine phosporibosyl transferase gene, CAD, the adenosine deaminase gene, the asparagine synthetase gene, the antibiotic resistance genes, e.g. tet^{r}, amp^{r}, Cm^{r} or cat, kan^{r} or neo^{r} (aminoglycoside phosphotransferase genes), the hygromycin B phosphotransferase gene, and the like.

In most embodiments, the linear expression cassette is a non-integrative polynucleotide, i.e., it does not integrate into a genome of a host cell, and, as such, typically does not contain recombination sites or flanking sequences to facilitate homologous recombination.

In certain embodiments, the heavy and light chain coding sequences are present on the same nucleic molecule, and expression of the two chains may be accomplished by using a single promoter and an internal ribosome entry site (IRES) between the two-coding sequences. Such constructs are known to those of skill in the art, see, e.g., Dirks, 1993 (Gene;128:247-9).

In most embodiments of the invention, an antibody producing cell, usually a single cell, is deposited into a well of a plate in a minimal volume (in about 0.1 μl, about 0.5 μl, about 1 μl, or about 5 μl), and polynucleotides encoding a immunoglobulin heavy chain variable domain and an immunoglobulin light chain variable domain are obtained, e.g. harvested, isolated, amplified, etc., isolated from the cell. In most embodiments this is done using an amplification procedure, such as the polymerase chain reaction.

For example, once an antibody-producing cell has been identified and plated, RNA is recovered from the cell by established methods, such as the method of Rappolee et al. (J. Cell Biochem. 39:1-11, 1989), or a scaled-down version of the method of Gonda et al. (J. Virol. 61.:2754-2763, 1987) and once RNA has been recovered, cDNA is made. Many methods for constructing cDNA from RNA are well known in the art, such as those described by Sambrook et al. (Sambrook, Fritsch and Maniatis, Molecular Cloning—A Laboratory Manual, Cold Spring Harbor Laboratory Press, 1989).

Sequences encoding heavy and light chains may be amplified from the cDNA using techniques well known in the art, such as Polymerase Chain Reaction (PCR). See Mullis, U.S. Pat. No. 4,683,195; Mullis et al., U.S. Pat. No. 4,683,195; Polymerase Chain Reaction: Current Communication in Molecular Biology, Cold Springs Harbor Press, Cold Spring Harbor, N.Y., 1989. Briefly, cDNA segments encoding the variable domain of the antibody are exponentially amplified by performing sequential reactions with a DNA polymerase. The reaction is primed by a 5′ and a 3′ DNA primer. In some embodiments, the 3′ antisense primer corresponding to a DNA sequence in the constant (or joining) region of the immunoglobulin chain and the 5′ primer (or panel of related primers) corresponding to a DNA sequence in the variable region of the immunoglobulin chain. This combination of oligonucleotide primers has been used in the PCR amplification of murine immunoglobulin cDNAs of unknown sequence (see Sastry et at., Proc Natl. Acad. Sci. 86:5728-5732, 1989 and Orlandi et al., Proc. Natl. Acad. Sci. 86:3833-3837, 1989). Alternatively, an “anchored polymerase chain reaction” may be performed (see Loh et al., Science 243:217-220, 1989). In this procedure, the first strand cDNA is primed with a 3′ DNA primer as above, and a poly(dG tail) is then added to the 3′ end of the strand with terminal deoxynucleotidyl transferase. The product is then amplified by PCR using the specific 3′ DNA primer and another oligonucleotide consisting of a poly(dC) tail attached to a sequence with convenient restriction sites. In many embodiments, however, the entire polynucleotide encoding a heavy or light chain is amplified using primers spanning the start codons and stop codons of both of the immunoglobulin cDNAs, however, depending on the amplification products desired, suitable primers may be used. Typical primers for use with rabbit antibody-producing cells are as follows: heavy chain, 5′ end (CACCATGGAGACTGGGCTGCGCTGGCTTCTCCTGGTCGCTGTG; SEQ ID NO: 1); heavy chain, 3′ end (CTCCCGCTCTCCGGGTAAATGAGCGCTGTGCCGGCGA; SEQ ID NO:2); light chain kappa, 5′end (CAGGCAGGACCCAGCATGGACACGAGGGCCCCCACT; SEQ ID NO:3); and L kappa, 3′end (TCAATAGGGGTGACTGTTAGAGCGAGACGCCTGC; SEQ ID NO:4). Suitable restriction sites and other tails may be engineered into the amplification oligonucleotides to facilitate cloning and further processing of the amplification products. Amplification procedures using nested primers may also be used, where such nested primers are well known to one of skill in the art.

Once polynucleotides encoding immunoglobulin heavy and light chain variable domains are amplified from a cell, they are assembled with appropriate antibody domains and/or regulatory sequences to form an expression cassette.

In order to assemble an expression cassette i.e. to operably link the coding sequences with any other coding or regulatory sequences, standard recombinant DNA technology (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.) may be used. Several methods are known in the art for producing antibody-encoding nucleic acids, including those found in U.S. Pat. Nos. 6,180,370, 5,693,762, 4,816,397, 5,693,761 and 5,530,101. One PCR method utilizes “overlapping extension PCR” (Hayashi et al., Biotechniques. 1994: 312, 314-5) to create expression cassettes for the heavy and light chain encoding nucleic acids. In this method multiple overlapping PCR reactions using the cDNA product obtained from the antibody producing cell and other appropriate nucleic acids as templates generates an expression cassette.

Depending on the constant regions and other regions used, several types of antibody that are known in the art may be made by this method. As well as full length antibodies, antigen-binding fragments of antibodies may be made. These fragments include, but are not limited to, Fab, Fab′ and F(ab′)₂, Fd, single-chain Fvs (scFv), single-chain immunoglobulins (e.g., wherein a heavy chain, or portion thereof and light chain, or portion thereof are fused), disulfide-linked Fvs (sdFv), diabodies, triabodies, tetrabodies, scFv minibodies, Fab minibodies; and dimeric scFv and any other fragments comprising a V_{L} and a V_{H} domain in a conformation such that a specific antigen binding region is formed. Antibody fragments, including single-chain antibodies, may comprise the variable region(s) alone or in combination with the entire or partial of the following: a heavy chain constant domain, or portion thereof, e.g., a CH1, CH2, CH3, transmembrane, and/or cytoplasmic domain, on the heavy chain, and a light chain constant domain, e.g., a Cₖₐₚₚₐ or C_{lambda} domain, or portion thereof on the light chain. Also included in the invention are any combinations of variable region(s) and CH1, CH2, CH3, Cₖₐₚₚₐ, C_{lambda}, transmembrane and cytoplasmic domains.

Production of circular vectors for expression of antibodies is well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.). Certain recombination-based methods, e.g. GATEWAY™ (InVitrogen, Carlsbad, Calif.)), CREATOR™ (Clontech, Palo Alto, Calif.) or ET cloning (Muyrers et al, Nucleic Acids Res. 27:1555-7 (1999)) methodologies may also be used in the production of expression cassettes.

Expression of Immunoglobulin Heavy and Light Chains

In most embodiments, a pair of immunoglobulin heavy and light chain expression cassettes are introduced directly into a host cell, and the cell incubated under conditions sufficient to induce expression of the encoded immunoglobulin heavy and light chains.

The pair of expression cassettes introduced into a host cell essentially maintains the combination of heavy and light chain-encoding nucleic acids that are present in the antibody cell from which the immunoglobulin variable domain-encoding nucleic acids of the expression cassettes are derived.

Any cell suitable for expression of expression cassettes may be used as a host cell. Usually, a mammalian host cell line that does not ordinarily produce antibodies is used, examples of which are as follows: monkey kidney cells (COS cells), monkey kidney CVI cells transformed by SV40 (COS-7, ATCC CRL 165 1); human embryonic kidney cells (HEK-293, Graham et al. J. Gen Virol. 36:59 (1977)); baby hamster kidney cells (BHK, ATCC CCL 10); chinese hamster ovary-cells (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1587): human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TRI cells (Mather et al., Annals N.Y. Acad. Sci 383:44-68 (1982)); NIH/3T3 cells (ATCC CRL-1658); and mouse L cells (ATCC CCL-1). Additional cell lines will become apparent to those of ordinary skill in the art. A wide variety of cell lines are available from the American Type Culture Collection, 10801 University Boulevard, Manassas, Va. 20110-2209.

Methods of introducing linear nucleic acids into cells are well known in the art. Suitable methods include electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995. In some embodiments lipofectamine and calcium mediated gene transfer technologies are used. Methods for introducing circular nucleic acids are also well known in the art and discussed in Ausubel, above.

After a pair of expression cassettes corresponding to the heavy and light chain nucleic acids isolated from a single antibody producing cell has been introduced into cell, the cells are typically incubated, normally at 37° C., sometimes under selection, for a period of about 1-24 hours in order to allow for the expression of the antibody. In most embodiment, the antibody is typically secreted into the supernatant of the media in which the cell is growing in.

The specificity of the antibodies produced by this method is unknown at the point at which they are expressed.

In an alternative embodiment, polynucleotides encoding at least the heavy and light chain variable domains of an antibody of an antibody producing cell are isolated, and introduced into a host cell prior to the modification of the polynucleotides into expression cassettes. In this embodiment, the polynucleotides are inserted into or may recombine with chromosomal or extrachromosomal host cell nucleic acids (either linear or circular e.g. a plasmid) to form expression cassettes and provide for expression of a monoclonal antibody. In some embodiments, the polynucleotides are modified to include e.g. recombination sequences for GATEWAY™ (InVitrogen, Carlsbad, Calif.), ET cloning (Muyrers et al, Nucleic Acids Res. 27:1555-7 (1999)); CREATOR™ (Clontech, Palo Alto, Calif.) or cre/lox or the like to facilitate. their insertion or recombination into host cell nucleic acids. In certain embodiments, a host nucleic acid and an immunoglobulin-encoding polynucleotide may be introduced together into the host cell, and an expression cassette is formed in the host cell.

Methods for Producing a Plurality of Monoclonal Antibodies

The invention provides methods for producing a plurality of monoclonal antibodies. In general, these methods involve performing the following steps on several individual antibody-producing cells: a) operably linking a first nucleic acid encoding an immunoglobulin heavy chain variable domain obtained from an antibody-producing cell to a second nucleic acid to form a expression cassette for an immunoglobulin heavy chain; b) operably linking a third nucleic acid encoding an immunoglobulin light chain variable domain obtained from the same antibody-producing cell to a fourth nucleic acid to form a expression cassette for an immunoglobulin light chain; (c) introducing the expression cassettes into a host cell; and (d) incubating the host cell under conditions sufficient to provide for production of an monoclonal antibody. Performing this method on each cell of a plurality of antibody producing cells, usually single cells, will result in the production of a plurality of monoclonal antibodies.

In many embodiments of the invention, the above methods for producing a monoclonal antibody are performed on a plurality of antibody-producing cells, each producing a different antibody. The plurality of antibody-producing cells are usually isolated from the same tissue (e.g. blood, spleen, lymph node, bone marrow etc.) and is usually isolated from the same animal.

In these methods, single antibody-producing cells are usually deposited into the wells of a multi-well plate. In general, 2, more than about 5, more than about 10, more than about 50, more than about 100, more than about 200, more than about 500, more than about 1000, more than about 2000, more than about 5000, or more than about 10,000 or more than about 50,000 cells, usually up to about 100,000 cells, are each individually deposited, usually into microtiter plates, which may be of a 24 well, 48 well, 96 well, 386 well or 1544 well format. In certain other embodiments, as described above more than a single cell may be deposited.

In many embodiments, primers are employed to obtain the desired coding sequences from the plurality of cells. Any convenient primer may be employed. In general, a single pair of degenerate primers, a single pair non-degenerate primers, a mixture of two, about four, about 6, about 8 or about 10 or more distinct primers of known sequence, or nested set of degenerate or non-degenerate primers is used to amplify the heavy chain variable domain-encoding nucleic acid from each of the individually deposited cells. In most embodiments, immunoglobulin heavy chain variable domain encoding polynucleotides can be amplified from more than about 10%, more than about 20%, more than about 50%, more than about 70%, more than about 80%, or more than about 90% of the individual cells plated.

In general, a single pair of degenerate or non-degenerate primers (or nested primer pair of degenerate or non-degenerate primers of known sequence) is used to amplify the light chain variable domain-encoding nucleic acid from each of the deposited cells. In certain embodiments, a single nested primer set of four non-degenerate primers of known sequence is used to amplify the light chain variable domain-encoding nucleic acid from each of the deposited cells. In most embodiments, immunoglobulin light chain variable domain encoding polynucleotides can be amplified from more than about 10%, more than about 20%, more than about 50%, more than about 70%, more than about 80%, or more than about 90% or more of the individual cells plated.

In many embodiments, amplification of the heavy and light chain variable domain-encoding polynucleotides occurs from several antibody-producing cells in parallel, generally depending on the size of the microtiter plate used. As such, amplification of these polynucleotides is usually performed in batches of 96, 384 or 1544.

In some embodiments, both the heavy and light chain polynucleotides are amplified in the same reaction vessel (i.e. for each cell, both the heavy and light chain polynucleotides are amplified in the same tube).

Keeping the amplification products separate from each other, for example in a 96-well plate, each amplification product is operably linked to other nucleic acid sequences to form an expression cassette, and each expression cassette is transferred into host cells, and antibodies are expressed and optionally secreted into culture media. As such, a plurality of monoclonal antibodies is made.

Compositions of the Invention

The invention further provides several compositions relating to monoclonal antibody production and screening.

In one embodiment, the invention provides a plurality of polynucleotide expression cassettes (linear or circular) for immunoglobulin heavy and light chain pairs for antibodies of unknown specificity. In other embodiments, the invention provides a plurality of host cells each containing a pair of subject expression cassettes, and a plurality of host cells each expressing an antibody of unknown specificity. In another embodiment, the invention provides a plurality of antibodies.

By plurality is meant at least 2, more than about 5, more than about 10, more than about 50, more than about 100, more than about 200, more than about 500, more than about 1000, more than about 2000, more than about 5000, or more than about 10,000 or more than about 50,000 or more, usually no more than about 100,000. These composition are usually contained in microtiter plates, which may be of a 24 well, 48 well, 96 well, 386 well or 1544 well format, and each plate is usually labeled with a unique identifier such that each sample will have a unique name, e.g. based on the name of the plate and the coordinates of the sample within the plate. These compositions may be referred to as “libraries”.

Methods of Screening a Plurality of Monoclonal Antibodies

The invention provides a method of screening a plurality of monoclonal antibodies. In general, this method involves producing a plurality of monoclonal antibodies using the method described above and screening the plurality of monoclonal antibodies using one or a combination of a variety of assays. In general, these assays are functional assays, and may be grouped as follows: assays that detect an antibody's binding affinity or specificity, and assays that detect the ability of an antibody to inhibit a process.

A monoclonal antibody identified as having a specific binding activity with an antigen, or an inhibitory activity is termed a monoclonal antibody of interest.

Binding Assays

In these assays, each antibody of a plurality of antibodies is tested for its ability to bind specifically to a substrate. The term “specifically” in the context of antibody binding, refers to high avidity and/or high affinity binding of an antibody to a specific antigen i.e., a polypeptide, or epitope. In many embodiments, the specific antigen is an antigen (or a fragment or subfraction of an antigen) used to immunize the animal host from which the antibody-producing cells were isolated. Antibody specifically binding an antigen or fragment thereof is stronger than binding of the same antibody to other antigens. Antibodies which bind specifically to a polypeptide may be capable of binding other polypeptides at a weak, yet detectable, level (e.g., 10% or less of the binding shown to the polypeptide of interest). Such weak binding, or background binding, is readily discernible from the specific antibody binding to a subject polypeptide, e.g. by use of appropriate controls. In general, specific antibodies bind to an antigen with a binding affinity of 10⁻⁷ M or more, e.g., 10⁻⁸ M or more (e.g., 10⁻⁹ M, 10⁻¹⁰, 10⁻¹¹, etc.). In general, an antibody with a binding affinity of 10⁻⁶ M or less is not useful in that it will not bind an antigen at a detectable level using conventional methodology currently used.

Typically, in performing a screening assay, antibody samples produced by a library of antibody producing host cells are deposited onto a solid support in a way that each antibody can be identified, e.g. with a plate number and position on the plate, or another identifier that will allow the identification of the host cell culture that produced the antibody.

The antibodies of the invention may be screened for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally involve lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4 degree. C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads).

Western blot analysis generally involves preparation of protein samples followed by electrophoresis of the protein samples in a polyacrylamide gel (e.g., 8%-20% SDS-PAGE depending on the molecular weight of the antigen), and transfer of the separated protein samples from the polyacrylamide gel to a membrane such as nitrocellulose, PVDF or nylon. Following transfer, the membrane is blocked in blocking solution (e.g., PBS with 3% BSA or non-fat milk), washed in washing buffer (e.g., PBS-Tween 20), and incubated with primary antibody (the antibody of interest) diluted in blocking buffer. After this incubation, the membrane is washed in washing buffer, incubated with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 1251), and after a further wash, the presence of the antigen may be detected. One of skill in the art would be knowledgeable as to the parameters that can be modified to increase the signal detected and to reduce the background noise.

ELISAs involve preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art.

The binding affinity of an antibody to an antigen and the off-rate of an antibody-antigen interaction can be determined by competitive binding assays. One example of a competitive binding assay is a radioimmunoassay comprising the incubation of labeled antigen (e.g., 3H or 125I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 1251) in the presence of increasing amounts of an unlabeled second antibody.

Antibodies of the invention may be screened using immunocytochemisty methods on cells (e.g., mammalian cells, such as CHO cells) transfected with a vector enabling the expression of an antigen or with vector alone using techniques commonly known in the art. Antibodies that bind antigen transfected cells, but not vector-only transfected cells, are antigen specific.

In certain embodiments, however, the assay is an antigen capture assay, and an array or microarray of antibodies may be employed for this purpose. Methods for making and using microarrays of polypeptides are known in the art (see e.g. U.S. Pat. Nos. 6,372,483, 6,352,842, 6,346,416 and 6,242,266).

Inhibitor Assays

In certain embodiments, the assay measures the specific inhibition of an antibody to an interaction between a first compound and a second compound (e.g. two biopolymeric compounds) or specifically inhibits a reaction (e.g. an enzymatic reaction). In the interaction inhibition assay, one interaction substrate, usually a biopolymeric compound such as a protein e.g. a receptor, may be bound to a solid support in a reaction vessel. Antibody is added to the reaction vessel followed by a detectable binding partner for the substrate, usually a biopolymeric compound such as a protein e.g. a radiolabeled ligand for the receptor. After washing the vessel, interaction inhibition may be measured by determining the amount of detectable binding partner present in the vessel. Interaction inhibition occurs when binding of the binding partner is reduced greater than about 20%, greater than about 50%, greater than about 70%, greater than about 80%, or greater than about 90% or 95% or more, as compared to a control assay that does not contain antibody.

In the reaction inhibition assay, an enzyme may be bound to a solid support in a reaction vessel. Antibody is usually added to the reaction vessel followed by a substrate for the enzyme. In many embodiments, the products of the reaction between the enzyme and the substrate are detectable, and, after a certain time, the reaction is usually stopped. After the reaction has been stopped, reaction inhibition may be measured by determining the level of detectable reaction product present in the vessel. Reaction inhibition occurs when the rate of the reaction is reduced greater than about 20%, greater than about 50%, greater than about 70%, greater than about 80%, or greater than about 90% or 95% or more, as compared to a control assay that does not contain antibody.

In vivo Assays

In certain embodiments the monoclonal antibodies are tested in vivo. In general, the method involves administering a subject monoclonal antibody to an animal model for a disease or condition and determining the effect of the monoclonal antibody on the on the disease or condition of the model animal. In vivo assays of the invention include controls, where suitable controls include a sample in the absence of the monoclonal antibody. Generally a plurality of assay mixtures is run in parallel with different antibody concentrations to obtain a differential response to the various concentrations. Typically, one of these concentrations serves as a negative control, i.e. at zero concentration or below the level of detection.

A monoclonal antibody of interest is one that modulates, i.e., reduces or increases a symptom of the animal model disease or condition by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, when compared to a control in the absence of the antibody. In general, a monoclonal antibody of interest will cause a subject animal to be more similar to an equivalent animal that is not suffering from the disease or condition. Monoclonal antibodies that have therapeutic value that have been identified using the methods and compositions of the invention are termed “therapeutic” antibodies.

Methods of Identifying Nucleic Acids Encoding a Monoclonal Antibody of Interest

The invention further provides a method of identifying a nucleic acid encoding a monoclonal antibody of interest. In general, the method involves: (a) immunizing an animal with an antigen;(b) producing a plurality of monoclonal antibodies derived from antibody producing cells of the animal by the methods described above; (c) screening the plurality of monoclonal antibodies to identify an monoclonal antibody of interest; and (d) identifying nucleic acids encoding the monoclonal antibody of interest.

Since the host cell expressing the antibody of interest contains the immunoglobulin heavy and light chain-encoding expression cassettes, the nucleic acids encoding the monoclonal antibody of interest may be identified if the host cell expressing the monoclonal antibody of interest is identified. As such, the subject nucleic acids may be identified by a variety of methods known to one of skill in the art. Similar methods are used to identify host cell cultures in monoclonal antibody production using hybridoma technology (Harlow et al., Antibodies: A Laboratory Manual, First Edition (1988) Cold spring Harbor, N.Y.).

For example, upon identifying a monoclonal antibody of interest, the host cell expressing the antibody of interest may be identified using a “look-up” table which lists, for every antibody sample, the corresponding host cell culture. In certain other embodiments, a look-up table containing antibody library sample identifiers, corresponding expression cassette library sample identifiers and/or host cell identifiers may be used to identify the subject nucleic acids.

Once identified, the nucleic acids encoding a monoclonal antibody of interest may be recovered, characterized and manipulated using techniques familiar to one of skill in the art (Ausubel, et al, Short Protocols in Molecular Biology, 3rd ed., Wiley & Sons, (1995) and Sambrook, et al, Molecular Cloning: A Laboratory Manual, Third Edition, (2001) Cold Spring Harbor, N.Y.).

Methods of Producing a Monoclonal Antibody of Interest

The invention provides several methods of producing a monoclonal antibody of interest. In general these methods involve incubating a host cell containing a nucleic acid encoding a monoclonal antibody of interest under conditions sufficient for production of the antibody. In many embodiments, the methods of producing a monoclonal antibody of interest involve transferring identified expression cassettes for an monoclonal antibody of interest into a suitable vector, and transferring the recombinant vector into a host cell to provide for expression of the monoclonal antibody. In some embodiments, the subject methods involve transferring at least the variable domain-encoding sequences from the identified heavy and light chains into vectors suitable for their expression in immunoglobulin heavy and light chains. Suitable constant domain-encoding sequences and/or other-antibody domain-encoding sequences may be added to the variable domain-encoding sequences at this point. These nucleic acid modifications may also allow for humanization of the subject antibody.

The subject monoclonal antibodies can be produced by any method known in the art for the synthesis of antibodies, in particular, by recombinant expression techniques.

Recombinant expression of a subject monoclonal antibody, or fragment, derivative or analog thereof, usually requires construction of an expression vector containing a polynucleotide that encodes the antibody. Methods which are well known to those skilled in the art can be used to construct expression vectors containing antibody coding sequences and appropriate transcriptional and translational control signals. These methods include, for example, in vitro recombinant DNA techniques and synthetic techniques. As such, the invention provides vectors comprising a nucleotide sequence encoding an antibody molecule of the invention.

The expression vector is transferred to a host cell by conventional techniques and the transfected cells are then cultured to produce a subject antibody. In most embodiments, vectors encoding both the heavy and light chains are co-expressed in the host cell to provide for expression of the entire immunoglobulin molecule.

A variety of host-expression vector systems may be utilized to express a subject monoclonal antibody. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells etc.) harboring recombinant expression constructs containing promoters derived from the genome of mammalian cells (e.g., metallothionein promoter) or from mammalian viruses (e.g., the adenovirus late promoter; the vaccinia virus 7.5K promoter). In many embodiments, bacterial cells such as Escherichia coli and eukaryotic cells are used for the expression of entire recombinant antibody molecules. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to , express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Aulographa californica nuclear polyhedrosis virus (AcNPV) is used as a vector to express antibodies. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized to express a subject antibody. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1 984)). The efficiency of expression may be enhanced by the inclusion of appropriate transcription enhancer elements, transcription terminators, etc. (see Bittner et al., Methods in Enzymol. 153:51-544 (1987)).

For long-term, high-yield production of recombinant antibodies, stable expression may be used. For example, cell lines, which stably express the antibody molecule may be engineered. Rather than using expression vectors which contain viral origins of replication, host cells can be transformed with immunoglobulin expression cassettes and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into a chromosome and grow to form foci which in turn can be cloned and expanded into cell lines. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Phamacol. Toxicol. 32:573-596 (1993); Mulligan, Science 260:926-932 (1993); and Morgan and Anderson, Ann. Rev. Biochem. 62:191-217 (1993); TIB TECH 11(5):155-215 (1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981).

The host cell may be co-transfected with two expression vectors of the invention, the first vector encoding a heavy chain derived polypeptide and the second vector encoding a light chain derived polypeptide. The two vectors may contain different selectable markers and origins of replication, which enable equal expression of heavy and light chain polypeptides. Alternatively, a single vector may be used which encodes, and is capable of expressing, both heavy and light chain polypeptides.

Once an antibody molecule of the invention has been produced, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In many embodiments, antibodies are secreted from the cell into culture medium and harvested from the culture medium.

Utility

The invention provides, inter alia, methods for producing a plurality of monoclonal antibodies, compositions containing a plurality of monoclonal antibodies, methods for screening a plurality of monoclonal antibodies, methods of identifying a monoclonal antibody of interest, and methods for expressing a monoclonal antibody of interest. These methods and compositions have several uses, many of which will be described below.

In one embodiment, the invention provides methods of treating a subject with a monoclonal antibody of interest. In general these methods involve administering a monoclonal antibody identified by the methods described above to a host in need of treatment. In many embodiments, the monoclonal antibody is a therapeutic monoclonal antibody.

By treatment is meant at least an amelioration of a symptom associated with the pathological condition afflicting the host, where amelioration is used in a broad sense to refer to at least a reduction in the magnitude of a parameter, e.g. symptom, associated with the pathological condition being treated. As such, treatment also includes outcomes where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts are treatable according to the subject methods. Generally such hosts are mammals or mammalian, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans. In other embodiment, the host will be an animal model for a human disease.

Of particular interest is treatment and prevention of diseases, conditions and disorders associated with abnormal expression of a cellular protein, usually present on the surface of a cell, e.g. a cancer cell.

The methods and compositions of the invention have several research applications. In one exemplary application, the library of monoclonal antibodies is deposited onto an array or microarray (e.g. using a method provided by U.S. Pat. Nos. 6,372,483, 6,352,842, 6,346,416 and 6,242,266), and labeled samples (e.g. cell extracts or proteins) or pairs of differentially labeled are incubated with the array. Such experiments may provide monoclonal antibodies and antibody-encoding polynucleotide sequences that differentially bind to samples. In one exemplary experiment, cancerous cells or extracts thereof are labeled and incubated with an array of monoclonal antibodies. After washing of the array, data representing the amount of binding of the cell or extract thereof may be extracted for each antibody. Comparison of this data to data generated using normal or non-cancerous cells incubated with a similar or the same array may reveal monoclonal antibodies that specifically recognize the cancer cell. Such antibodies have therapeutic applications.

The methods and compositions of the invention provide specific reagents that can be used in standard diagnostic procedures. For example, the antibodies or their immunoreactive fragments can be employed in immunoassays for detection of target antigens. To perform a diagnostic method, on of the compositions of the invention is provided as a reagent to detect a target antigen in a sample with which it reacts. Procedures for performing immunoassays are well established in the art and hence are not described here.

The human monoclonal antibodies generated by the subject methods may also be used for treatment or prevention of diseases and conditions. The monoclonal antibodies may be used to modulate the activities of target antigens that play a central role in disease development and/or progression. For example, a humanized anti-Her2 antibody, available commercially under the trademark HERCEPTIN®, which selectively inhibits growth of human breast cancer cells, is now employed as a potent drug to treat tens and thousands of breast cancer patients who overexpress the breast cancer antigen Her2.

Kits

Also provided by the subject invention are kits for practicing the subject methods, as described above. The subject kits at least include one or more of: a plurality (i.e. about 2, more than about 5, more than about 10, more than about 50, more than about 100, more than about 500, more than about 1000, more than about 5000, more than about 10,000, more than about 20,000 or more than about 50,000, usually up to about 100,000) of monoclonal antibodies, linear expression cassettes containing monoclonal antibody encoding polynucleotides or cells containing pairs of the polynucleotides; or degenerate or non-degenerate oligonucleotide primer pairs or nested primers sets for amplifying immunoglobulin heavy and light chain variable domain-encoding polynucleotides. Other optional components of the kit include: components for performing antibody binding assays, e.g. microtiter plates and ELISA reagents; buffers, nucleotides and reagents for performing amplifying heavy and light chain nucleic acids; and antibodies and reagents for enriching for antibody producing cells. The various components of the kit may be present in separate containers or certain compatible components may be precombined into a single container, as desired.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Also provided by the subject invention is are kits including at least a computer readable medium including programming as discussed above and instructions. The instructions may include installation or setup directions. The instructions may include directions for use of the invention with options or combinations of options as described above. In certain embodiments, the instructions include both types of information.

Providing the software and instructions as a kit may serve a number of purposes. The combination may be packaged and purchased as a means for producing rabbit antibodies that are less immunogenic in a non-rabbit host than a parent antibody, or nucleotide sequences them.

The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc, including the same medium on which the program is presented.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Direct Expression of IgG cDNAs Derived from a Rabbit Hybridoma

As a model antibody, the B1 rabbit mAb was used which is a high-affinity antibody to the alpha-v beta-6 integrin. PCR primer were designed based on the 5′ and 3′ sequences of rabbit IgG H and L chains (SEQ ID NOS:1-4), and used to perform RT-PCR with B1 hybridoma cell lysates as a template. Bands of the expected size were obtained for both H and L chains. We then joined the H and L chain PCR products with CMV promoter and bovine growth hormone (BGH) 3′ terminator segments, by using overlap extension PCR as described below. PCR products of the expected size were observed, suggesting that the H and L chain expression cassettes had been formed correctly. The linear H and L expression cassettes were then used for co-transfecting HEK-293 cells, using lipofectamine 2000 to promote DNA uptake. After two days, supernatants were collected from the transfected cells, and tested for antibody activity in a cell-ELISA assay (staining of beta-6 integrin transfected HEK-293 cells). HRP-coupled secondary antibody was used for detection.

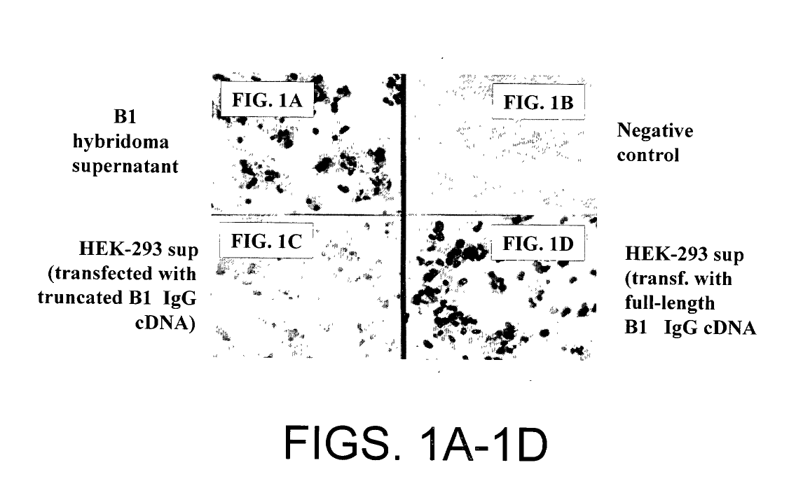

FIG. 1 shows the results of the cell-ELISA assay: cell-ELISA analysis of anti-beta-6 integrin antibody activity in experimental cultures supernatant of the B1 hybridoma (A), or of HEK-293 cells transfected with linear expression cassettes containing B1 IgG L+truncated H cDNA (C), or full-length B1 H+L cDNAs (B and D). HEK-293 cells transiently transfected with beta-6 cDNA (A, C, D) or mock-transfected cells (B) were plated in 96-well plates, fixed, and stained with the antibody containing supernatants. Note the similarity in staining pattern and intensity between the original B1 antibody (A) and its recombinant form (D). Also note that the full-length recombinant B1 antibody strongly stains beta-6 transfected cells (D), but does not stain mock-transfected cells (B). Panel A shows staining of the beta-6 transfected cells with the B1 antibody (hybridoma supernatant). Note the typical staining of transient transfectants, with some cells expressing at high levels, while others are negative. As shown in Panel D, an identical staining pattern was obtained with the supernatant from the B1 IgG-transfected HEK-293 cells (recombinant IgG). This shows that the antibody was assembled and processed correctly in the transfected cells, and was expressed at a level similar to hybridoma cells. We also tested a truncated IgG product corresponding to the F(ab′)2 fragment (Panel C). The fragment produced detectable. Panel B shows a negative control (reaction of mock-transfected HEK-293 cells with the HEK-293 supernatant containing full-length recombinant B1 IgG). In other words, the same recombinant antibody that produced strong staining of beta-6 transfected cells (panel D), did not produce any staining of mock-transfected cells. This shows that the recombinant antibody is specifically reacting with the beta-6 integrin, in a manner that is indistinguishable from the original hybridoma supernatant.

These results show that linear IgG expression cassettes produced by PCR can be co-expressed in HEK-293 cells, and can direct secretion of monoclonal antibody at levels comparable to hybridoma cell supernatants.

### Example 2

### Analysis of Bone Marrow Cells from Immunized Rabbits

To test whether plasma cells in the bone marrow of immunized rabbits are detectable, cells were collected from the femur and tibia of an immunized rabbit. After red cell lysis, the cells were washed, suspended in PBS, fixed with paraformaldehyde and permeabilized with Triton X-100. Cells were then analyzed by flow cytometry with FITC-labeled goat-anti-rabbit IgG. We found that most of the cells were negative, while a small population (approx. 1.5%) were strongly positive. Similarly, when FITC-labeled cells were spread on a microscope slide and inspected by fluorescence microscopy, we counted 5 brightly stained cells in a field of approximately 250 cells. When bone marrow cells from a non-immunized rabbit were analyzed in an identical way, the percentage of brightly stained cells was about 5 times lower than in the immunized rabbit. This experiment shows that we can detect bone marrow plasma cells by IgG staining, and that there are increased numbers of plasma cells in immunized rabbits.

### Example 3

### IgG-Secreting Plasma Cells in the Bone Marrow of Immunized Rabbits

Immunization: Human plasma fibronectin purified by gelatine-Sepharose chromatography is commercially available. New Zealand white rabbits are immunized by subcutaneous injection of 200 micrograms of fibronectin, dissolved in 0.5 ml of PBS and emulsified with an equal volume of TiterMax™ Gold adjuvant (available from Sigma-Aldrich Co.). The immunogen is divided up and injected into multiple sites along the back and legs. A maximum of 0.1 ml is injected into each site. For booster immunization; a smaller total amount (100 micrograms) of immunogen is used. Booster immunizations are given at 3-week intervals, and antibody titers are determined 10 days after each boost. 2 booster injections are usually sufficient for obtaining high antibody titer.

Collection of bone marrow cells: 9 days after each booster injection, the serum titer of anti-fibronectin antibodies is determined by ELISA. As soon as a high titer (detectable signal at 1:100,000 dilution) is observed, the rabbit is sacrificed and bone marrow cells are collected from the femur and/or other large bones. Spleen cells and peripheral blood mononuclear cells (PBMCs) are also collected and frozen in 10% DMSO/90% FBS for analysis at a later time. We have previously determined that rabbit lymphocytes survive freezing under these conditions and can be used for generating hybridomas, with no loss of fusion efficiency. Very large numbers of bone marrow cells (>2 billion) are obtained from a single rabbit. After washing, clearing of debris, and red-cell lysis, cells are kept as single-cell suspensions, and analyzed as described below. Cells are collected from 4 rabbits total: 2 rabbits immunized with fibronectin, and 2 non-immune rabbits.

Analysis of bone marrow plasma cells: To determine the percentage of plasma cells, and the percentage of fibronectin-specific plasma cells, bone marrow cells are analyzed in three different ways:

Flow cytometry: As a plasma cell marker, an antibody to the cell-surface proteoglycan, syndecan-1 (mAb281-2) (Brauker et al., 1991 Dev Biol 147:285-92 and Sanderson et al., 1989 Cell Regul 1:27-35) is used. This is a rat mAb raised against mouse syndecan-1, which cross-reacts with rabbit syndecan-1. It is well established that in the bone marrow, plasma cells are the only cells expressing syndecan-1. This marker is not found on memory cells or proliferating B cell blasts in lymphoid organs. The mAb 281-2 antibody is available from B D Pharmingen (San Diego, Calif.), and was previously shown to react with rabbit plasma cells. Staining of rabbit bone marrow cells with this antibody, followed by PE-labeled anti-rat antibody and FACS analysis, provides a means to quantitate the percentage of plasma cells in the bone marrow of immunized vs. non-immune rabbits. In addition, cells are stained with FITC-labeled goat anti-rabbit IgG antibody. This is done with unpermeabilized cells, to detect cell-surface IgG, as well as permeabilized cells, to detect intracellular IgG. In a preliminary experiment, we have done this analysis and found that after permeabilization, a population (approx. 1%) of very brightly staining cells was detected. Two-color analysis is also performed, to confirm that the population of cells staining brightly with anti-IgG is the same as the one stained by anti-syndecan antibody.

Cytospin analysis: Bone marrow cells are spun onto a slide, and stained with anti-syndecan-1 and anti-rabbit IgG antibodies, as described above. Stained slides are observed by immunofluorescence microscopy and phase contrast microscopy, to show that the plasma cells identified by antibodies possess the typical morphological characteristics of plasma cells. Positive cells are counted on microscopic images, to determine the percentage of plasma cells in the total bone marrow cell population.

Analysis for production of anti-fibronectin (ELISPOT assay). Bone marrow cells are plated on dishes coated with a mixture of collagen (5 micrograms per ml) and fibronectin (1 microgram per ml), followed by blocking with 1% BSA. Plasma cells are expected to adhere to the dish, since it is known that plasma cells adhere well to collagen. Non-adherent cells are removed by gentle rinsing with PBS, and the cells are overlaid with soft agar and serum-free cell culture medium. Dishes are incubated at 37° C. for 5 hours, to allow secreted anti-fibronectin antibodies to bind to the fibronectin coated on the dish. The dishes are then be washed, and probed with HRP-conjugated anti-rabbit IgG antibody. HRP are detected using an insoluble substrate. Microscopic inspection of the dishes determines the number of plasma cells that produce anti-fibronectin antibody.

The combination of syndecan-1 staining and anti-IgG staining should permit accurate determination of plasma cell numbers in the bone marrow. Furthermore, the comparison between immunized rabbits and pre-immune rabbits will provide an estimate of the percentage of plasma cells that are part of the immune response to fibronectin. The percentage of cells producing anti-fibronectin antibody will also be detemined directly, using the ELISPOT assay.

### Example 4

### Isolating Rabbit Plasma Cells and Sorting them at Single-Cell Density into Multiwell Plates

The cells obtained in example 3 are processed using two different methods:

a) Flow cytometry followed by single-cell plating. Cell populations obtained from bone marrow are incubated either with the anti-syndecan-1 antibody, mAb281-2 (see above), or with PE-labeled anti-rabbit IgG.

Labeled cells are sorted using a FACSVantage SE cell sorter (Becton-Dickinson, San Jose, Calif.). This instrument has the capacity to rapidly sort large numbers of cells (24,000 events per second) and single-cell dispense the positive cells into 96-well plates. Cells are directly sorted into 96-well plates containing RT-PCR buffer, and subjected to RT-PCR with nested primers specific for the IgG heavy and light chains. The FACSVantage SE system has already been used for a very similar experiment. In this experiment, antigen-reactive CD4⁺ T cells were sorted by five-color flow cytometry, and their T-cell receptor sequences were determined following single-cell PCR.

b) Plating of plasma cells in 384-well plates at limiting dilution. As an alternative to cell sorting, limiting dilution plating is used in order to obtain single plasma cells. The percentage of plasma cells in the harvested cell suspension are estimated according to previous experiments. For example, bone marrow cell suspensions are likely to contain 1 to 2% plasma cells. Thus, bone marrow suspensions are plated in 384-well plates at a density of 20 cells per well. On the average, 10 to 20% of the wells will contain a single plasma cell, and only a very small percentage of wells will contain more than one plasma cell. The expected number of plasma cells per plate are between 38 and 76. All of the wells are subjected to RT-PCR with nested primers specific for the IgG heavy and light chains, using a total volume of 10 microliters per well. The presence of other bone marrow cells should not interfere with the amplification of plasma cell IgG cDNA, since plasma cells are the only cells in bone marrow that express secreted IgG. Memory B cells, which may also reside in the bone marrow, express membrane-bound IgG, which will not be amplified by the primers we are using. The presence of other cells may even be advantageous, because the mRNA from these cells will act as a carrier for the very small amount of mRNA present in a single plasma cell.

### Example 5

RT-PCR to Obtain Heavy and Light Chain cDNAs from Single Plasma Cells

Primer design: In rabbit, the 5′ coding sequences of rabbit immunoglobulin heavy chain are primarily derived from only one gene. Antibody diversity is created by gene conversion and somatic mutation, but this does not affect the 5′ end of the antibody cDNA. Thus, most rabbit IgG H chains have very similar or identical signal peptide sequences, and the same is true for L chains. On the 3′ side, we will use primers hybridizing to the constant domains, which also have identical sequences in most rabbit antibodies (rabbit constant domains are not divided into subclasses). As a result, only one pair of primers each is required for amplifying the vast majority of rabbit IgG H and L sequences. Typical priming sites are shown below, although any primer sites are used so long as the a variable domain-encoding polynucleotide is amplified. Typical primers for use with rabbit antibody-producing cells are as follows: heavy chain, 5′ end (CACCATGGAGACTGGGCTGCGCTGGCTTCTCCTGGTCGCTGTG (SEQ ID NO:1); heavy chain, 3′ end (CTCCCGCTCTCCGGGTAAATGAGCGCTGTGCCGGCGA (SEQ ID NO:2); light chain kappa, 5′end (CAGGCAGGACCCAGCATGGACACGAGGGCCCCCACT); and L kappa, 3′end (TCAATAGGGGTGACTGTTAGAGCGAGACGCCTGC).

Note that the 3′ H chain primer spans the 3′ end of the coding region, the stop codon, and the beginning of the 3′ UTR. Thus, this primer is specific for the secreted form of IgG, and does not recognize the transmembrane form, which does not contain this sequence due to alternative splicing. Therefore, the method is unlikely to recover IgG from memory B cells, which express predominantly the transmembrane form.

RT-PCR conditions: For single-cell PCR, it may be necessary to perform RT-PCR directly from the cell lysate, without purifying mRNA. We have successfully used protocols that effect cell lysis by heating, which is immediately followed by reverse transcription and PCR. To maximize efficiency, a reagent kit is used specifically designed for RT-PCR from small numbers of cells (cells-to-cDNA kit, Ambion, Austin Tex.). The procedure involves cell lysis by heating in a buffer containing RNAse inhibitors, followed by DNA degradation and reverse transcription performed at high temperature (60° C.). Reverse transcription is primed either by oligo-dT or by primers specific for the 3′ region of the IgG mRNAs. A single-step RT-PCR protocol is used, utilizing a thermostable enzyme that has both reverse transcriptase and DNA polymerase activities (MasterAmp™ RT-PCR Kit for High Sensitivity, Epicentre Technologies, Madison, Wis.). PCR products are analyzed by agarose gel electrophoresis. If required, a second round of PCR is performed with nested primers. In some PCR applications, this step is required to produce sufficient amounts of specific product.

Co-amplification of H and L chain cDNAs: Different combinations of primers are tried, to accomplish efficient PCR amplification of H and L chain cDNAs in the same reaction. A ‘head start’ approach is often used, where PCR cycling is started with H chain primers alone; after a number of cycles (5 to 10) the L chain primers are added to the mix. Using these methods, similar yields of H and L chain are produced. Alternatively, a nested PCR approach is used for the H chain, by performing an initial round of PCR with primers amplifying the full-length cDNA, and a second round with primers amplifying only the vH-cH1-hinge portion of the H chain. This method should yield a product similar in size to the L chain cDNA. Expression of this product yields the F(ab′)2 fragment of IgG, which is divalent and fully active for antigen-binding.

Minituarization: PCRs are performed in 1536-well, 384-well or 96-well plates, with a reaction volume of 2.5 to 10 microliters/well. For 384-well plates, we use a Whatman/Biometra T1 cycler (LabRepco, Horsham, Pa.), which is specifically designed for efficient PCR. Alternatively, we use a BioOven III hot-air thermocycler (St. John Associates, Inc, Beltsville, Md.). This instrument is designed for high throughput PCR, with a capacity of six 1536-well plates.

### Example 6

### Combining Heavy and Light Chain cDNAs with Promoter and Terminator Sequences for Expression in Mammalian Cells

IgG heavy and light chain PCR products generated as described in example 5 are joined with CMV promoter and BGH3′pA (bovine growth hormone polyadenylation/transcription termination) sequences. In initial experiments, IgG cDNA amplified from a previously characterized rabbit hybridoma are used as a positive control. This hybridoma (termed B1) produces a high-affinity antibody to the alpha-v beta-6 integrin.

Method a) Overlap Extension PCR.

CMV promoter segment: To prepare the CMV promoter fragment, the expression vector pcDNA-3 (which contains the CMV promoter and BGH3′pA segments) is used as a template, and the following PCR setup:

Primer 1 (5′′AATTCACATTGATTATTGAG 3′; SEQ ID NO:5) corresponding to the 5′end of the CMV promoter;

Primer 2 (5′ CAGCGCAGCCCAGTCTCCATCCCGTAAGCAGTGGGTTCTC 3′; SEQ ID NO:6) corresponding to the 3′ end of the CMV promoter, and containing a 5′ extension (underlined) complementary to the 5′ end of the rabbit Ig H signal peptide sequence is performed.

PCR amplification with these primers produces a linear DNA fragment consisting of the CMV promoter (610 nt) and a 20 nt extension on the 3′ end, which is complementary to the 5′ end of the IgG vH coding region. As mentioned above, most rabbit IgGs contain 5′ vH (signal peptide) regions with nearly identical sequences. Therefore, only one primer pair is needed to amplify the majority of rabbit IgG cDNAs.

BGH3′pA segment. A similar approach is used to prepare the BGH3′pA segment. Again, the pcDNA3 expression vector is used as a template, and the following primers are used:

Primer 3 (5′CCGGGTAAATGAGCGCTGTGGTTTAAACCCGCTGATCAGC 3′; SEQ ID NO:7), corresponding to 5′ end of the BGH3′pA domain extended by a 20 nt sequence complementary to the 3′ end of the IgG heavy chain coding region, and including 11 nt of the 3′ untranslated domain.

Primer 4 (5′ AAGCCATAGAGCCGACCGCA 3′; SEQ ID NO:8) corresponding to the 3′ end of the BGH polyadenylation domain.

PCR amplification results in a 250 nt fragment containing the BGH3′pA sequence and a 20 nt extension that overlaps with the 3′ end of the IgG heavy chain sequence.

Overlap extension PCR: The IgG heavy chain PCR product are mixed with the CMV promoter and BGH3′pA segments. The mixture are subjected to 10 cycles of PCR. The overlapping segments anneal, followed by extension of the overlapping 3′ ends. At the end of the 10 cycles, the outside primers (primers 1 and 4) are added to the mixture, and another 30 cycles of PCR are performed. The product is a 2100 nt fragment consisting of the CMV promoter, the IgG H coding sequence, and the BGH terminator.

IgG light chain: The process are carried out in an analogous manner to produce 1500 nt fragments consisting of CMV promoter, kappa light chain coding sequence, and BGH terminator. The majority of rabbit IgG antibodies contain kappa light chains; therefore, it will likely not be necessary to design a separate set of primers for lambda light chains.

A low concentration of primers in the initial PCR reaction may be used. In many embodiments, primers are be designed such-that amplification of the heavy chain results in a nucleotide encoding a form of the IgG H chain that is truncated at the 3′ end of the hinge domain. This fragment would be similar in size to the v kappa light chain. Co-expression of these fragments results in the secretion of F(ab′)₂ fragments of IgG.

Method b) Topoisomerase I Coupling.

This method is used as an alternative to overlap extension PCR. The overall experimental strategy is as described above. Commercially available topoisomerase-modified CMV promoter and BGH3′pA segments will be used (Invitrogen, San Diego, Calif.). The CMV promoter element (610 nt) is provided in a modified form with the topoisomerase recognition site (CCCTT) at its 3′ end, and a six base pair single-stranded overhang at the 3′ end (GCCTTG) which is used for directional coupling with the PCR product. The topoisomerase I enzyme is bound to the recognition site CCCTT. In order to be joined to the Topo-modified CMV promoter, the PCR product needs to contain the sequence CGGAACAAGGG (SEQ ID NO:9) at its 5′end. This sequence is cleaved by topoisomerase, resulting in a 6-base single-strand overhang that is complementary to the single-strand overhang of the CMV promoter element. These overhangs anneal and the fragments are covalently joined by the enzyme.

In order to link the IgG cDNA fragment to the CMV promoter, the 5′ primer used in the last round of IgG amplification are extended at its 5′ end with the sequence CGGAACAAGGG (SEQ ID NO:10).

The linkage of the 3′ end of the IgG fragment with the BGH3′pA element is performed in an analogous manner, except that a different single-stranded overhang (GACTCA) is being used. This provides for directionality and selective joining of the 5′ end with the CMV promoter and the 3′ with the BGH terminator.

The joining reaction is carried out by mixing the 5′ CMV element, IgG PCR product, and 3′BGH element at a 1:2:1 ratio, and adding the 10× reaction buffer. The reaction proceeds rapidly and is usually complete within 10 min at room temperature. Following the reaction, a secondary PCR reaction is carried out, using primers corresponding to the 5′ end of the CMV promoter and the 3′ end of the BGH terminator (primers 1 and 4, see above). This results in the formation of the 2.1 kb IgG H expression cassette, or the 1.5 kb IgG L expression cassette. After the reaction conditions have been optimized for H and L chains separately, we will establish conditions for co-amplification of H and L cassettes in the same reaction.

Conditions for co-production of H and L IgG expression cassettes in the same reaction are also envisioned.

### Example 7

### Expression of Monoclonal Antibodies in Mammalian Cells

Transfections are carried out either in 96-well plates or in 384-well plates. Extended PCR products containing the H and L expression cassettes are transferred to fresh plates, and the original PCR plates are stored at −20° C. to provide a permanent source for each antibody cDNA. The transferred DNA is mixed with either lipofectamine 2000 (Invitrogen) or polyethylenimine (ExGen 500 transfection reagent; Fermentas Inc., Hanover, Md). Cells (CHO, HEK-293, or 240E) suspended in goat serum-containing medium are added to the reagent-DNA mixture. The cells are then incubated at 37° C. for 2 days. Removal of the transfection reagents is unnecessary, because both reagents are non-toxic to the cell lines used. At the end of the incubation period, samples are taken from the cell supernatants and tested for the presence of rabbit IgG antibodies, using either one of the following assays:

Sandwich ELISA assay. Multiwell plates are coated with goat-anti rabbit IgG, followed by incubation with transferred supernatants from the transfected cells. Bound antibody is detected with HRP-coupled goat anti-rabbit IgG. This assay is very sensitive and detects IgG-producing transfectants even if the amount of rabbit IgG produced is very low.

Antigen-binding assay. In experiments using antibodies derived from known hybridoma cells, antibody activity is detected using either ELISA with the purified antigen, or cell-ELISA with transfected cells expressing the antigen. In the case of our previously used positive control, the B1 antibody to the alpha-v beta-6 integrin, cells transfected with the beta-6 subunit are used. These cells are plated in multiwell plates, fixed, and incubated with samples of the IgG-transfected cell supernatants. Binding of antibody is detected with HRP-conjugated goat anti-rabbit antibodies. In experiments with transfected antibody cDNAs derived from plasma cells of rabbits immunized with fibronectin, antigen-binding activity are detected in a standard ELISA assay, using purified fibronectin as the antigen.

Rapid high-throughput methods for purifying PCR products are available (e.g., QuiaQuick 96 PCR purification kit; Quiagen, Valencia, Calif.) and may be used.

It is evident from the above results and discussion that the subject invention provides an important new means for generating monoclonal antibodies. Specifically, the subject invention provides a system for generating nucleic acids encoding a plurality of monoclonal antibodies, a plurality of cells containing the nucleic acids, and a plurality of monoclonal antibodies. As such, the subject methods and systems find use in a variety of different applications, including research, therapeutic and other applications. Accordingly, the present invention represents a significant contribution to the art.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method of screening a plurality of monoclonal antibodies, said method comprising:
(a) producing a plurality of immunoglobulin heavy and light chain expression cassette pairs from a plurality of different antibody producing cells that produce antibodies of unknown specificity, each expression cassette pair comprising:
(i) a first nucleic acid encoding an immunoglobulin heavy chain variable domain derived from an antibody-producing cell operably linked to a second nucleic acid form an immunoglobulin heavy chain expression cassette, and
(ii) a third nucleic acid encoding an immunoglobulin heavy chain variable domain derived from said antibody-producing cell operably linked to a fourth nucleic acid form an immunoglobulin light chain expression cassette, and
(b) introducing each immunoglobulin heavy and light chain expression cassette pair of said plurality of immunoglobulin heavy and light chain expression cassette pairs into a different host cell;
(c) incubating said host cells under conditions sufficient to provide for production of a plurality of monoclonal antibodies; and
(d) screening said monoclonal antibodies.

2. The method according to claim 1, wherein said first nucleic acid encodes an entire immunoglobulin heavy chain or fragment thereof.

3. The method according to claim 1, wherein said third nucleic acid encodes an entire immunoglobulin light chain or fragment thereof.

4. The method according to claim 1, wherein said antibody-producing cell is selected from a plasma cell, a B cell and a hybridoma cell.

5. The method according to claim 1, wherein said antibody-producing cell is a plasma cell isolated from bone marrow.

6. The method according to claim 1, wherein said antibody-producing cell is from a rabbit and said method is a method for screening a plurality of rabbit monoclonal antibodies.

7. The method according to claim 1, wherein said second nucleic acid encodes at least one of a heavy chain constant region, a promoter, and a transcriptional terminator

8. The method according to claim 7, wherein said fourth nucleic acid encodes at least one of a light chain constant region, a promoter, and a transcriptional terminator.

9. The method according to claim 1, wherein said constant region-encoding nucleic acids of said second and forth nucleic acids are constant region-encoding nucleic acids native to said antibody-producing cell.

10. The method according to claim 1, wherein said expression cassette for an immunoglobulin heavy chain and said expression cassette for an immunoglobulin light chain are linked on the same nucleic acid molecule.

11. The method according to claim 1, wherein said plurality of monoclonal antibodies is a plurality of monoclonal antibodies of unknown specificity.

12. The method according to claim 1, wherein said cell is from an animal immunized with an antigen.

13. The method according to claim 1, wherein said immunoglobulin heavy chain variable domain-encoding nucleic acid and said immunoglobulin light chain variable domain-encoding nucleic acid are obtained using nucleic acid amplification.

14. The method according to claim 13, wherein said nucleic acid amplification using a single pair of oligonucleotide primers.

15. The method according to claim 1, wherein said host cell is a mammalian host cell.

16. The method according to claim 1, wherein said immunoglobulin heavy and light chain expression cassettes are linear immunoglobulin heavy and light chain expression cassettes.

17. The method of claim 1, wherein said plurality of monoclonal antibodies is 50 or more monoclonal antibodies.

18. The method according to claim 1, wherein said screening step is screening said plurality of monoclonal antibodies for binding specificity to said antigen.

19. The method according to claim 1, wherein said screening step is screening said plurality of monoclonal antibodies for binding specificity to an epitope of said antigen.

20. The method according to claim 1, wherein said screening step is screening said plurality of monoclonal antibodies for a therapeutic antibody.

21. The method according to claim 1, wherein said screening step is screening said plurality of monoclonal antibodies for a inhibitory monoclonal antibody that inhibits an interaction between a first biopolymeric compound and a second biopolymeric compound.

22. The method according to claim 1, wherein said screening step (c) is screening said plurality of monoclonal antibodies for an inhibitory monoclonal antibody that inhibits an enzymatic reaction.

23. A method of identifying a monoclonal antibody of interest, said method comprising:
(a) performing the method of claim 1; and
(b) identifying a monoclonal antibody of interest.

24. A method of identifying a nucleic acid encoding a monoclonal antibody of interest, said method comprising:
(a) performing the method of claim 23; and
(b) identifying a nucleic acid encoding said monoclonal antibody of interest.
